(19) United States
(12) Patent Application Publication
(10) Pub. No.: US 2008/0097347 A1
(43) Pub. Date: Apr. 24, 2008

(54) Bendable needle assembly

(76) Inventor: Babak Arvanaghi, Mclean, VA (US)

Correspondence Address: BERENATO, WHITE & STAVISH, LLC, 6550 ROCK SPRING DRIVE, SUITE 240, BETHESDA, MD, 20817, US

(21) Appl. No.: 11/525,134

(22) Filed: Sep. 22, 2006

(51) Int. Cl. A61M 5/158

(52) U.S. Cl. 604/264

(57) ABSTRACT

The needle assembly comprises a needle including an elongated tubular needle body having open distal and proximal ends, and an elongated stylet having a solid shank complementary to the tubular needle body and slidably movable therewithin substantially coaxially thereto. The needle body includes a tip portion defining the distal end and a main portion defining the proximal end. Moreover, the tip portion bendable relative to the main portion. In turn, the stylet shank has a solid distal end. Furthermore, at least one of the distal end of the needle body and the distal end of the stylet shank is beveled at an angle to a longitudinal axis of corresponding one of the needle body and the stylet shank. The beveled distal end exerts a bending force to the tip portion of the needle body against the beveled face during advancement of the needle body through a human tissue.

### BACKGROUND OF THE INVENTION

1. Field of the Invention

This invention pertains in general to needle assemblies and, more particularly, to a needle assembly including a tubular needle and a stylet complementary to the tubular needle and provided for controlling an angle of inclination of a tip portion of the needle.

2. Description of the Prior Art

A large number and variety of medical needle assemblies, such as biopsy needles, epidural needles, or the like, are available and in widespread use for performing various medical procedures including but not limited to biopsy, epidural injection, disc decompression or the like. Typically, these procedures have been carried out using a hollow (tubular) needle of varying internal diameter with a tapered distal end and diagonal cutting edge. The tubular needle has the open distal end forming an axial port for placing the open distal end of the needle in target areas of a human tissue. A rod-like stylus is inserted within the hollow needle and the assembly is then inserted into a patient's body (human tissue) until the tissue to be removed is engaged. When the distal end of the needle has reached the target area of the tissue, the stylus is removed from the needle.

The above medical needle assemblies of the prior art are subject to certain disadvantages. For instance, as the current tubular needles are straight or curved and made of a substantially rigid (non-deformable and non-pliable) material, they have been unable to precisely position the open distal end of the needle in target areas of the human tissue, such as by bending. Therefore they have not been useful in application for hard to reach locations in the body, while minimally invasive medical procedures are preferred in many cases. Thus, the distal ends of the current needle assemblies are typically positioned where is accessible with rigid straight or curved needles, as opposed to where is needed. Accordingly, there is room for improvement of the medical needle assemblies for advancing the art.

### SUMMARY OF THE INVENTION

The present invention is directed to a novel medical needle assembly provided to precisely position a distal end thereof in a target area of a human tissue.

The needle assembly according to the present invention comprises a needle including an elongated tubular needle body having open distal and proximal ends, and an elongated stylet having a solid shank complementary to the tubular needle body and slidably movable therewithin substantially coaxially thereto. The needle body includes a tip portion defining the distal end and a main portion defining the proximal end. Moreover, the tip portion bendable relative to the main portion. In turn, the stylet shank has a solid distal end. Furthermore, at least one of the distal end of the needle body and the distal end of the stylet shank is beveled at an angle to a longitudinal axis of corresponding one of the needle body and the stylet shank. The at least one beveled distal end of one of the needle body and the stylet shank exerts a bending force to the tip portion of the needle body against the beveled face during advancement of the needle body through a biological (human) tissue. As a result, the stylet controls an angle of inclination of the tip portion of the needle body relative to the main portion thereof in order to steer the needle during advancement thereof through the biological tissue.

Therefore, the present invention provides a novel needle assembly allowing for controlled steering of the needle assembly as it advances through the biological tissue in order to position a distal end of a needle exactly where needed as opposed to where is accessible with straight needles.

### BRIEF DESCRIPTION OF THE DRAWINGS

FIG. 1 is an exploded perspective view of a needle assembly in accordance with a first exemplary embodiment of the present invention;

FIG. 2 is a cross-sectional view of a needle according to the first exemplary embodiment of the present invention;

FIG. 3 is a cross-sectional view of a first (rigid) stylet of the present invention:

FIG. 4 is a cross-sectional view of a second (pliable) stylet of the present invention;

FIG. 5 is a cross-sectional view of the needle assembly according to the first exemplary embodiment of the present invention with the first (rigid) stylet;

FIG. 6A is a sectional view of the needle assembly according to the first exemplary embodiment of the present invention (with the first (rigid) stylet) penetrating a human tissue;

FIG. 6B is a sectional view of the needle assembly according to the first exemplary embodiment of the present invention (with the second (pliable) stylet) advancing and bending through the human tissue;

FIG. 6C is a sectional view of the needle according to the first exemplary embodiment of the present invention with the first (rigid) stylet partially pulled back from a needle body;

FIG. 7 is an exploded perspective view of a needle assembly in accordance with a second exemplary embodiment of the present invention;

FIG. 8 is a cross-sectional view of the needle assembly according to the second exemplary embodiment of the present invention with the second (pliable) stylet having a beveled face completely extending outwardly from a distal face of a needle body;

FIG. 9A is a sectional view of the needle assembly according to the second exemplary embodiment of the present invention (with the first (rigid) stylet) penetrating a human tissue;

FIG. 9B is a sectional view of the needle assembly according to the second exemplary embodiment of the present invention (with the second (pliable) stylet) advancing and bending through the human tissue, shown as missing a target area of the human tissue;

FIG. 9C is a sectional view of the needle according to the second exemplary embodiment of the present invention (with the second (pliable) stylet) advancing and bending through the human tissue, shown with changed orientation of a beveled face thereof for correcting its trajectory;

FIG. 10 is a cross-sectional view of the needle assembly according to the second exemplary embodiment of the present invention with the second (pliable) stylet having the beveled face partially extending outwardly from the distal face of the needle body;

FIG. 11 is an exploded perspective view of a needle assembly in accordance with a third exemplary embodiment of the present invention;

FIG. 12 is a cross-sectional view of a needle according to the third exemplary embodiment of the present invention;

FIG. 13A is a sectional view of the needle assembly according to the third exemplary embodiment of the present invention (with the rigid stylet) penetrating the human tissue;

FIG. 13B is a sectional view of the needle according to the third exemplary embodiment of the present invention with the rigid stylet partially pulled back from a needle body so that a distal end of the stylet lies in a first flexible hinge portion of the needle body;

FIG. 13C is a sectional view of the needle according to the third exemplary embodiment of the present invention with the rigid stylet partially pulled back from a needle body so that a distal end of the stylet lies in a third flexible hinge portion of the needle body;

FIG. 14 is a cross-sectional view of a needle according to a fourth exemplary embodiment of the present invention with a beveled distal end;

FIG. 15 is a cross-sectional view of the needle according to alternative arrangement of the fourth exemplary embodiment of the present invention without an orthogonal distal end;

FIG. 16 is a cross-sectional view of a needle according to a fifth exemplary embodiment of the present invention.

### DESCRIPTION OF THE PREFERRED EMBODIMENTS

The preferred embodiments of the present invention will now be described with the reference to accompanying drawing.

For purposes of the following description, certain terminology is used in the following description for convenience only and is not limiting. The words such as “outermost” and “innermost”, “inwardly” and “outwardly”, “left” and “right” designate directions in the drawings to which reference is made. The words “smaller” and “larger” refer to relative size of elements of the apparatus of the present invention and designated portions thereof. The terminology includes the words specifically mentioned above, derivatives thereof and words of similar import. Additionally, the word “a”, as used in the claims, means “at least one”.

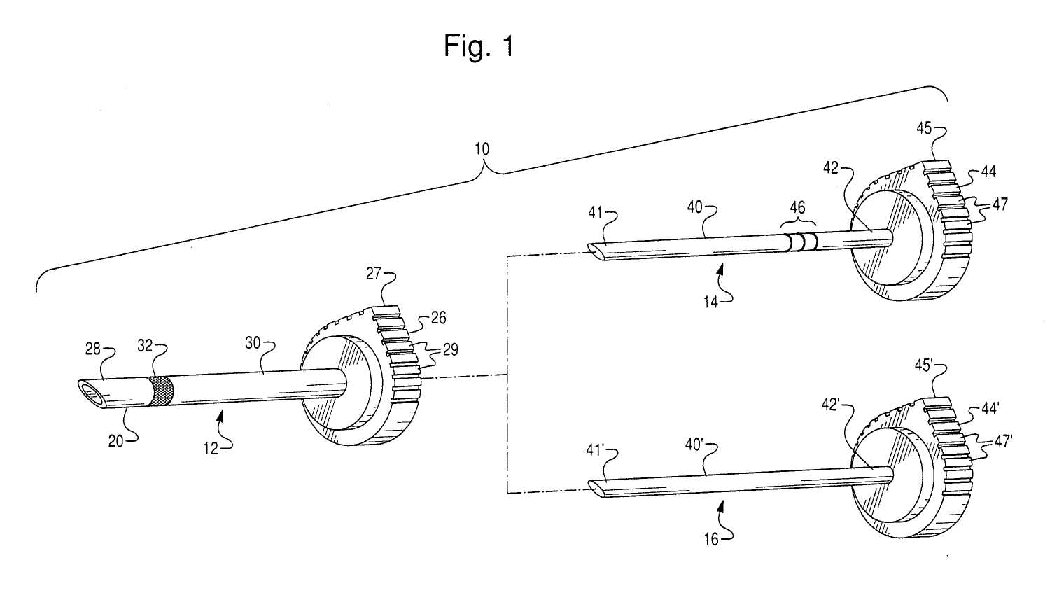

The present invention relates to a bendable needle assembly, such as a biopsy needle, epidural needle, or the like. FIGS. 1 and 5 of the drawings depict a first exemplary embodiment of the needle assembly of the present invention generally denoted by reference numeral 10.

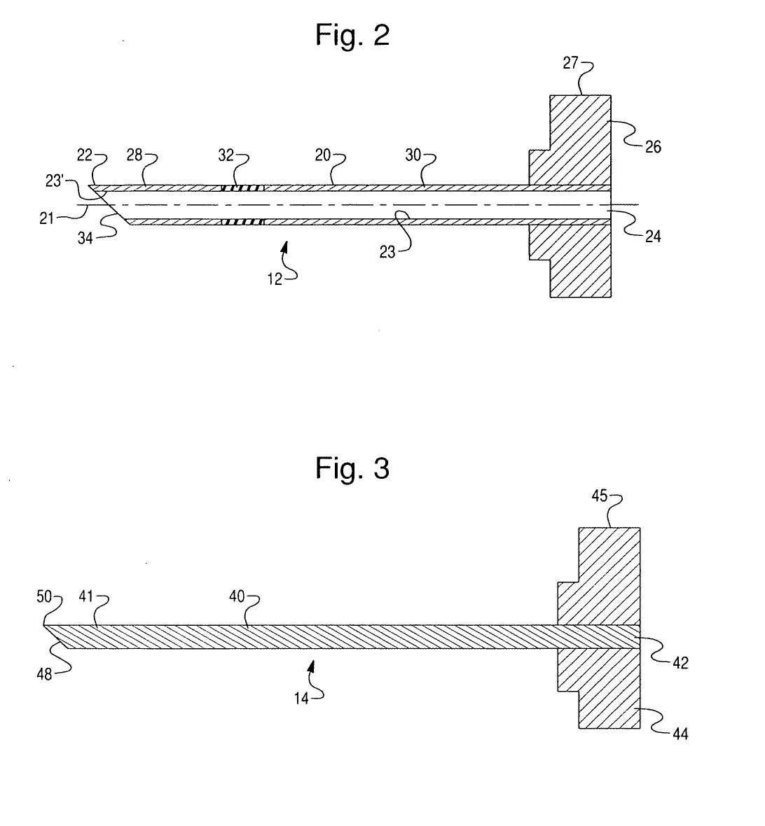

The needle assembly 10 comprises a needle 12, a first elongated stylet 14 and a second elongated stylet 16. As illustrated in FIGS. 1 and 2, the needle 12 has an elongated tubular, preferably cylindrical, needle body 20 having open distal and proximal ends 22 and 24, respectively, and a needle hub (or needle handle) 26 provided at the proximal end 24. The needle body 20 has a central longitudinal axis 21 and includes a rigid, penetrating tip portion 28 defining the distal end 22 thereof, a rigid main portion 30 defining the proximal end 24, and a bendable (pliable) hinge portion 32 connecting the tip portion 28 to the main portion 30 so as to enable the tip portion 28 to bend relative to the main portion 30. Preferably, the rigid tip portion 28 and the rigid main portion 30 of the needle body 20 are made of a conventional steel material of medical needles. The bendable hinge portion 32 is made of any appropriate pliable material, such as pliable plastic material of conventional catheters. Distal ends of the hinge portion 32 are connected to corresponding ends of the tip portion 28 and the main portion 30 in any appropriate manner known in the art, such as by adhesive bonding.

According to the first exemplary embodiment of the present invention, the distal end 22 of the tip portion 28 of the needle body 20 is beveled at an acute angle to the longitudinal axis 21 of the needle body 20 to form a beveled (oblique) planar oval face 34 extending across a width of the needle body 20. The beveled face 34 of the distal end 22 of the needle body 20 defines a sharp knife edge 36 allowing the tip portion 28 of the needle 12 more readily penetrate biological tissue. The beveled face 34 extends from an end 36 along a length of the needle body 20 to a tip 37 of the distal end 22 of needle body 20. The tip 37 is defined as an outermost point of the needle body 20. The acute angle between the beveled face 34 and the longitudinal axis 21 of the needle body 20 is preferably in the range of 30-60°.

The needle hub 26 of the needle 12 in accordance with the present invention is asymmetrical having a pointed ridge 27 oriented in alignment with the tip 37 of the beveled face 34 of the distal end 22 (or tip portion 28) of the needle body 20. Such an arrangement allows a tactile confirmation of the bevel direction, i.e. location of the tip 37 of the beveled face 34, and further allows for better grasping and avoidance of unintentional twisting of the needle 12 when a luer lock is being attached to the needle hub 26. Preferably, the needle hub 26 of the needle 12 is made of a transparent material, such as clear plastic, to see blood flashbacks, and is provided with a plurality of ridges 29 to allow for good grasping by a hand of a user.

The first stylet 14 illustrated in FIGS. 1 and 3, includes an elongated solid shank 40 having solid distal and proximal ends 41 and 42, respectively, and a stylet hub (or stylet handle) 44 attached to the proximal end 42 of the stylet shank 40. The stylet shank 40 of the first stylet 14 is made of rigid (hard and stiff), substantially non-pliable material, such as metal or hard plastic. As illustrated in FIG. 5, the stylet shank 40 is complementary to an interior cylindrical surface 23 of the tubular needle body 20 and is provided to be slidably movable therewithin in the direction between the distal and proximal ends 22, 24 of the needle body 20. As further illustrated in FIG. 5, the stylet shank 40 is received in the tubular needle body 20 substantially coaxially thereto. The distal end 41 of the stylet shank 40 of the first stylet 14 is beveled at an acute angle to a longitudinal axis of the stylet shank 40 to form a beveled (oblique) planar oval face 48 extending across a width of the stylet shank 40. The acute angle between the beveled face 48 and the longitudinal axis of the stylet shank 40 is substantially complementary to the bevel angle of the beveled face 34 of the distal end 22 of the needle body 20 and is preferably in the range of 30-60°. The beveled face 48 of the distal end 41 of the stylet shank 40 includes a tip 50 of the first stylet 14. The tip 50 is defined as an outermost point of the stylet shank 40.

The stylet hub 44 of the first stylet 14 in accordance with the present invention is asymmetrical having a pointed ridge 45 oriented in alignment with the tip 50 of the beveled face 48 of the distal end 41 of the stylet shank 40. Such an arrangement allows a tactile confirmation of the bevel direction, and further allows for better grasping and avoidance of unintentional twisting of the first stylet 14. Preferably, the stylet hub 44 of the first stylet 14 is provided with a plurality of ridges 47 to allow for good grasping by the hand of the user.

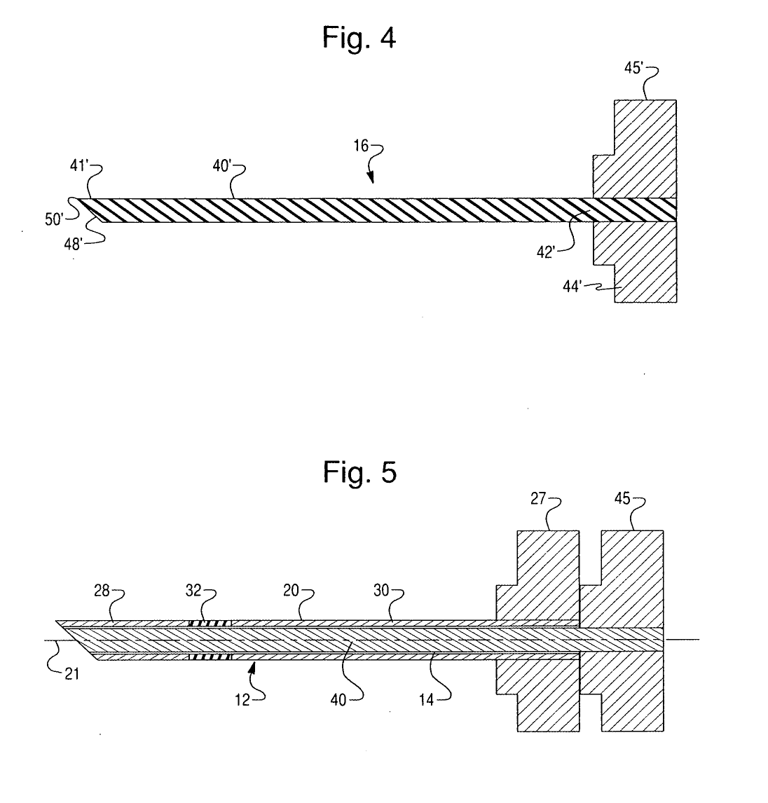

The second stylet 16 illustrated in FIGS. 1 and 4, is geometrically substantially identical to the first stylet 14, and includes an elongated solid shank 40′ having solid distal and proximal ends 41 and 42′, respectively, and a stylet hub (or stylet handle) 44′ attached to the proximal end 42′ of the stylet shank 40′. Consequently, the stylet shank 40′ is complementary to the interior cylindrical surface 23 of the tubular needle body 20 and, like the stylet shank 40 of the first stylet 14, is provided to be slidably movable within the needle body 20 in the direction between the distal and proximal ends 22, 24 thereof substantially coaxially thereto. Moreover the distal end 41′ of the stylet shank 40′ of the second stylet 16 is beveled at an acute angle to a longitudinal axis of the stylet shank 40′ to form a beveled (oblique) planar oval face 48′ extending across a width of the stylet shank 40′. The acute angle between the beveled face 48′ and the longitudinal axis of the stylet shank 40′ is substantially complementary to the bevel angle of the beveled face 34 of the distal end 22 of the needle body 20 and is preferably in the range of 30-60°. The beveled face 48′ of the distal end 41′ of the stylet shank 40′ includes a tip 50′ of the second stylet 16. The tip 50′ is defined as an outermost point of the stylet shank 40′. However, unlike the first stylet 14, the stylet shank 40′ of the second stylet 16 is made of a relatively soft, pliable (bendable) material, such as bendable plastic material.

The stylet hub 44 of the second stylet 16 in accordance with the present invention is asymmetrical having a pointed ridge 45′ oriented in alignment with the tip 50′ of the beveled face 48′ of the distal end 41′ of the stylet shank 40′. Such an arrailgnment allows a tactile confirmation of the bevel direction, and further allows for better grasping and avoidance of unintentional twisting of the second stylet 16. Preferably, the stylet hub 44′ of the second stylet 16is provided with a plurality of ridges 47′ to allow for good grasping by the hand of the user.

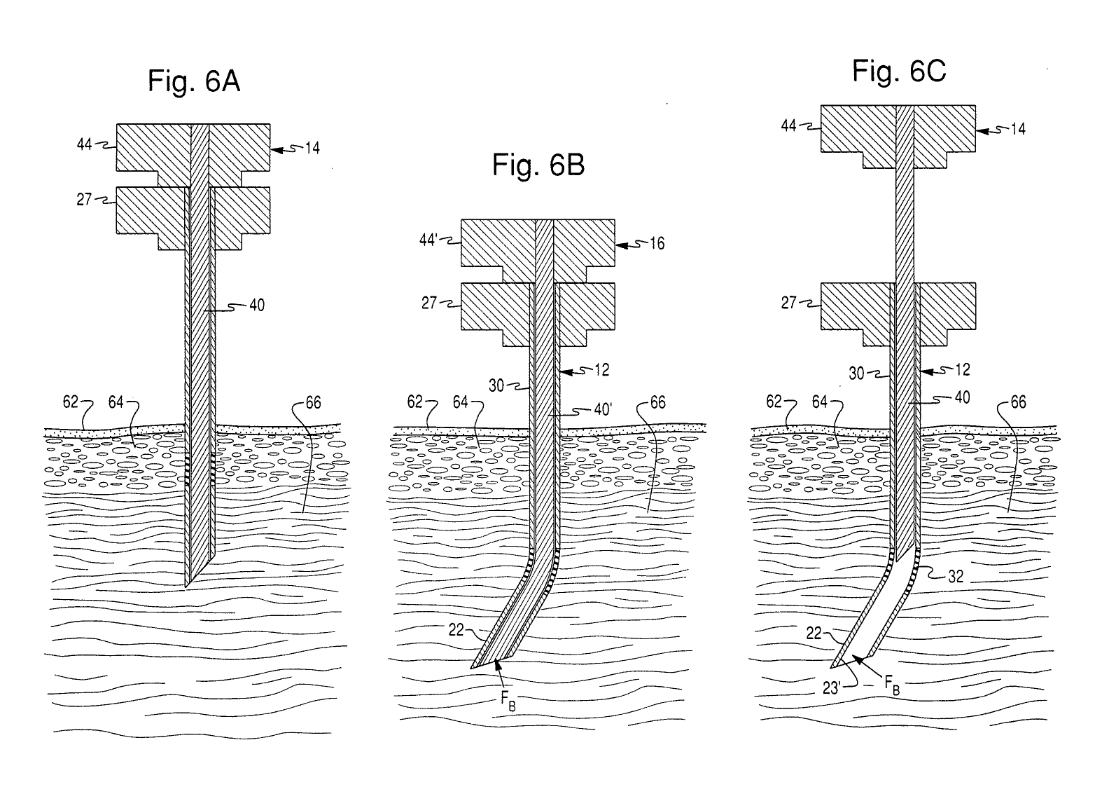

In operation, the needle assembly 10 is first inserted into a biological (human) tissue 60 including a skin 62, a loose tissue 64 and a muscle tissue 66, shown in FIG. 6A. In order to insert the needle 12 through the skin 62, the first (rigid) stylet 14 is used, as illustrated in FIG. 6A. The first (rigid) stylet 14 is inserted into the needle body 20 of the needle 12 so that the beveled face 48 of the stylet shank 40 is adjacent to, preferably substantially in flush with, the beveled face 34 of the needle body 20. Alternatively, the tip 50 of the beveled face 48 of the stylet shank 40 may slightly extend outwardly from the beveled face 34 of the needle body 20 or spaced inwardly therefrom. As the needle assembly 10 advances through the human tissue 60, resistance against the beveled face 48 of the first stylet 14 to the advancement of the needle 12 through the human tissue 60 exerts a bending force to the tip portion 28 of the needle body 20 against the beveled face 48 of the first stylet 14 and the beveled face 34 of the needle 12. However, the first (rigid) stylet 14 prevents the needle 12 from bending, thus allowing the needle 12 to penetrate the skin 62.

Once at an appropriate depth, the hard stylet 14 is removed from the needle 12 and replaced with the second (pliable) stylet 16 that allows the bending (deflection) of the tip portion 28 of the needle 12 when it is advanced thru the tissue 60, as illustrated in FIG. 6B. Specifically, as the needle assembly 1 0 advances through the human tissue 60, resistance against the beveled face 48′ of the second stylet 16 and the beveled face 34 of the needle 12 to the advancement of the needle 12 through the human tissue 60 applies a bending force F_{B} against the beveled face 48′ and the beveled face 34. The bending force F_{B} applied against the beveled faces 34 and 48′ is thus exerted to the tip portion 28 of the needle body 20. Thus, as the stylet shank 40′ of the second stylet 16 is pliable, lateral deflection of the tip portion 28 of the needle body 20 will occur due to the bending of the flexible hinge portion 32 against the beveled face 34 of the needle 12 by the bending force F_{B} applied (acting) to the beveled face 48′ of the pliable stylet 16 and the beveled face 34 of the needle 12 as the needle assembly 10 advances through the tissue 60, as illustrated in FIG. 6B. One of ordinary skill in the art would understand that the softer the stylet shank 40′ of the second stylet 16, the bigger the angle of deflection of the tip portion 28 relative the main portion 30 of the needle 12. Therefore, the stylets 14 and 16 of the needle assembly 10 control an angle of inclination of the tip portion 28 of the needle body 20 relative to the main portion 30 thereof.

Alternatively, the needle assembly 10 may include only one, rigid stylet 14. In operation, first, the rigid stylet 14 is inserted into the needle body 20 of the needle 12 so that the beveled face 48 of the stylet shank 40 is adjacent to the beveled face 34 of the needle body 20. Next, the needle assembly 10 is inserted through the skin 62 and advanced to the appropriate depth within the human tissue 60. Then, if the need to change the direction of the tip portion 28 of the needle body 20 arises (in order to steer the needle 12 in a desired direction), the rigid stylet 14 is partially pulled back from the needle body 20 (away from the beveled face 34 of the needle body 20) until the distal end 41, hence the tip 50, of the stylet shank 40 of the stylet 14 lies in the flexible hinge portion 32 of the needle body 20. It will be appreciated that further advancing of the needle 12 will cause the deflection (or bending) of the tip portion 28 of the needle body 20 against the beveled face 34 of the needle 12, as shown in FIG. 6C. Specifically, as the needle assembly 10 advances through the human tissue 60, resistance to the advancement of the needle 12 through the human tissue 60 applies a bending force F_{B} against an internal surface 23′ of the beveled distal end 22 of the needle body 20 adjacent to the tip 37 thereof. The bending force F_{B} applied against the beveled distal end 22 of the needle body 20 is thus exerted to the tip portion 28 of the needle body 20. Thus, as the hinged portion 32 of the needle body 20 is pliable, lateral deflection of the tip portion 28 of the needle body 20 will occur due to the bending of the flexible hinge portion 32 against the beveled distal end 22 of the needle 12 by the bending force F_{B} applied (acting) to the distal end 22 of the needle 12 as the needle assembly 10 advances through the tissue 60, as illustrated in FIG. 6C. Placing one or more depth markers/grading 46 on the proximal end 36 of the stylet shank 40 of the rigid stylet 14 (shown in FIG. 1) would let the user, such as injectionist, know as to when the tip 50 of the stylet 14 is in the flexible hinge portion 32 of the needle body 20. Therefore, the stylet 14 of the needle assembly 10 allows for controlling the angle of inclination of the tip portion 28 of the needle body 20 relative to the main portion 30 thereof.

Therefore, the needle assembly of the present invention, including the tubular needle with deflectable tip portion, the rigid stylet and the soft, flexible stylet, provides an inexpensive and economical alternative for the regular injections (Foraminal blocks, Lumbar sympathetics, and celiac plexus blocks) and performs better than any current needle assembly available for any of medical procedures.

Most blocks, and disc access procedures are easier with the needle having the deflectable tip portion. The needle assembly 10 of the present invention allows a slight paramedian approach to the disc or any other target as opposed to the traditional paramedian approach. This way, with very little training, all surgeons will be able to access any disc, even the very degenerated L5-SI disc with a long inter-facet distance, and a high pelvis. With the needle assembly of the present invention, the injectionist should be able to easily avoid an emerging nerve root, and be able to access a center of the disc or its posterior capsule without any difficulty. Accessing the appropriate target in the disc is important for good discography, and decompression of the disc.

For discography and insertion of any catheter or intra-discal device, the length of the tip portion 28 of the needle body 20 and the hinge portion 32 have to be optimized to get the tip 37 of the needle 12 into the center of the disc. Preferably, in the application for discography, the hinge portion 32 is about 1 to 1½ inch from the tip 37 of the needle body 20.

The needle assembly 10 of the present invention will also work for selective epidural injections (transforaminal or SNRB), posterior epidural insertion of spinal cord stimulators (cephalad, or caudad placements), Lumbar sympathetic blocks, and all RF procedures. Interlaminar thoracic epidural access will also be a lot easier with this type of device. Even a novice injectionist will be able to access the center of any disc by lining up the endplates, advancing the needle 12 with the hard stylet 14 to the posterior edge of the disc (not shown) just paramedian to it. Then the stylet 14 is changed to the softer stylet 16. Further advancing the needle 12 will cause the tip portion 28 thereof to bend against its beveled face 34, and enter the disc and reach its center, while avoiding the nerve root. The change in the stylet (soft versus hard) will also decrease the incidence of discitis.

As for spinal cord stimulators, a tuohy needle (a hollow needle, very slightly curved at the end. suitable for inserting epidural catheters) could help with cephalad or caudad placement of the leads. The cephalad positioning is rather easy with a regular tuohy needle, but the caudad placement of the leads in the sacrum (for pelvic pain, Interstitial cystitis, etc.) is very challenging. The needle assembly of the present invention should make the caudad placement of the stimulator lead very easy as it helps to guide the tip portion of the needle in a caudad direction. It should also help with the thoracic placement of the tip portion of the needle which at times is challenging.

Radiofrequency of the lumbar facet nerves will also be easier with t the needle assembly 10 of the present invention. For this procedure, the needle 12 is advanced to the junction of the transverse process and the pars (not shown). The stylet 14 is pulled back, and the needle 12 is advanced. The tip portion 28 of the needle 12 will follow against its bevel face 34 to lie against a groove, perfectly along a facet dorsomedial branch. Lumbar sympathetic blocks and celiac plexus blocks and its contributories should also be easier with the needle assembly of the present invention.

The needle assembly of the present invention may also substantially improve the current techniques for percutaneous disc decompression. The needle assembly of the present invention allows a much better placement of a decompression device exactly where needed as opposed to where is accessible with straight needles. By placing the decompressing device at the bulge or along a line connecting it to the center of the disc, the results of decompression improve.

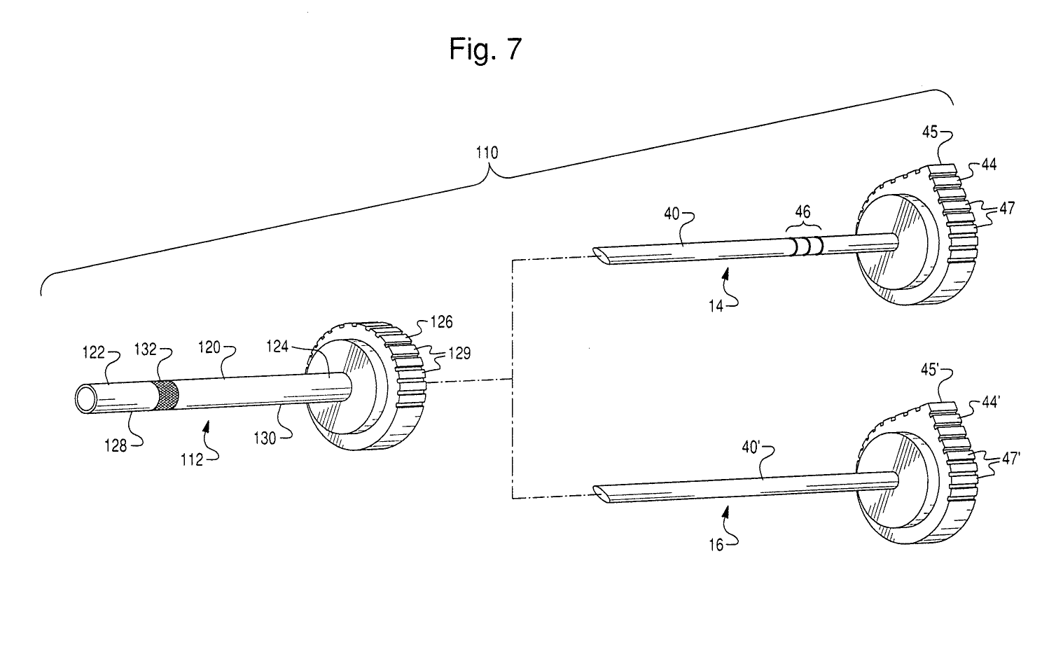

FIGS. 7-10 illustrate a needle assembly 110 according to a second exemplary embodiment of the present invention. Components, which are unchanged from the previous exemplary embodiments of the present invention are labeled with the same reference characters. Components, which function in the same way as in the first exemplary embodiment of the present invention depicted in FIGS. 1-6C are designated by the same reference numerals to which 100 has been added, sometimes without being described in detail since similarities between the corresponding parts in the two embodiments will be readily perceived by the reader.

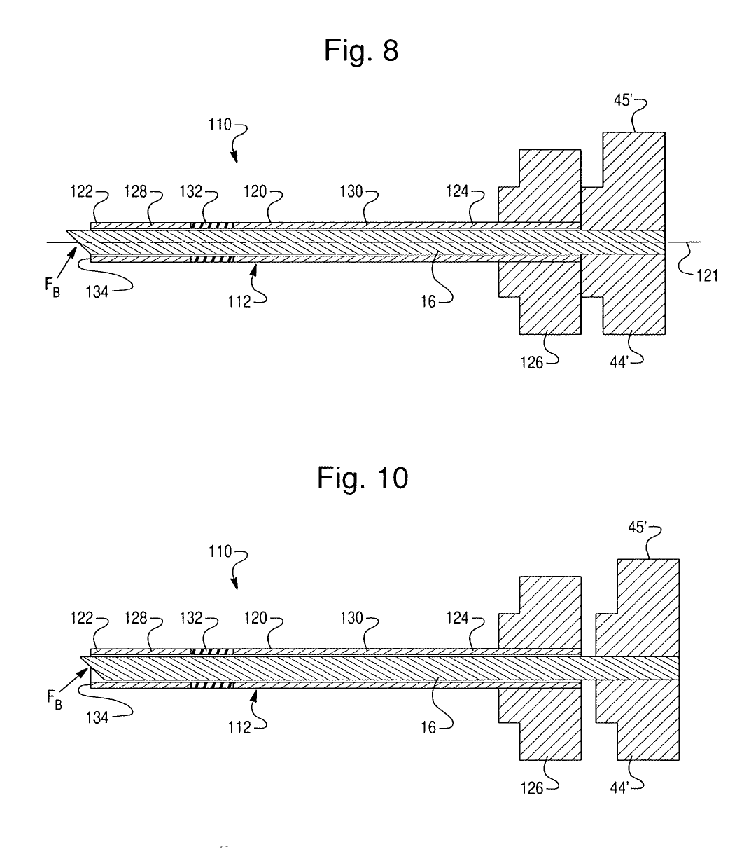

The needle assembly 110 according to the second exemplary embodiment of the present invention comprises a needle 112, a first elongated stylet 14 and a second elongated stylet 16. As illustrated in FIGS. 7 and 8, the needle 112 has an elongated tubular, preferably cylindrical, needle body 120 having open distal and proximal ends 122 and 124. respectively, and a needle hub (or needle handle) 126 provided at the proximal end 124. Preferably, the needle hub 126 of the needle 112 is made of a transparent material, such as clear plastic, to see blood flashbacks, and is provided with a plurality of ridges 129 to allow for good grasping by a hand of a user. The needle body 120 has a central longitudinal axis 121 and includes a rigid tip portion 128 defining the distal end 122 thereof, a rigid main portion 130 defining the proximal end 124, and a flexible hinge portion 132 connecting the tip portion 128 to the main portion 130 so as to enable the tip portion 128 to bend relative to the main portion 130. Preferably, the rigid tip portion 128 and the rigid main portion 130 of the needle body 120 are made of a conventional steel material of medical needles. The flexible hinge portion 132 is made of any appropriate pliable material, such as pliable plastic material of conventional catheters. Distal ends of the hinge portion 132 are connected to corresponding ends of the tip portion 128 and the main portion 130 in any appropriate manner known in the art, such as by adhesive bonding. Further according to the second exemplary embodiment of the present invention, the distal end 122 of the tip portion 128 of the needle body 120 is symmetrical and without a bevel. In other words, a distal face 134 of the needle body 120 is substantially orthogonal to the longitudinal axis 121 thereof the needle body 120.

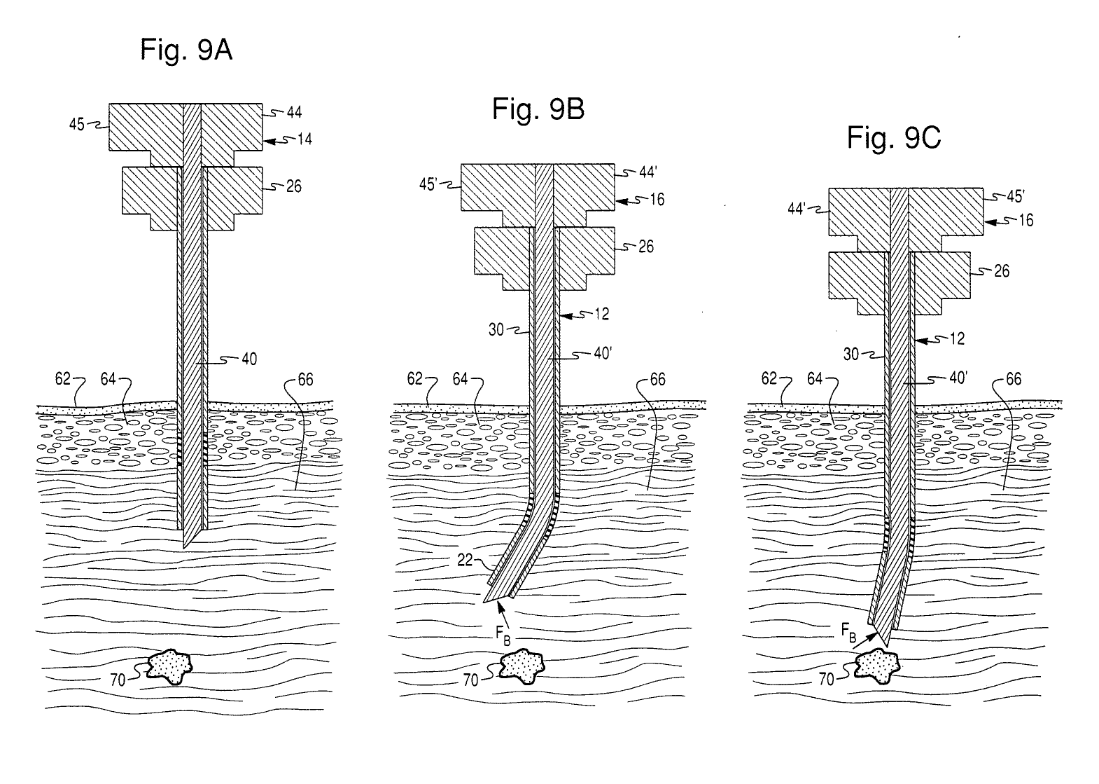

In operation, the needle assembly 110 is first inserted into the human tissue 60. In order to insert the needle 12 through the skin 62, the first (rigid) stylet 14 is used, as illustrated in FIG. 9A. The first (rigid) stylet 14 is inserted into the needle body 120 of the needle 112 so that the beveled face 48 of the stylet shank 40 completely extends outwardly from the distal face 134 of the needle body 120, as shown in FIG. 9A.

Once at an appropriate depth, the hard stylet 14 is removed from the needle 12 and replaced with the second (pliable) stylet 16 that allows the bending (deflection) of the tip portion 128 of the needle 112 when it is advanced thru the tissue 60, as illustrated in FIG. 9B. Specifically, as the needle assembly 1 10 advances through the human tissue 60, resistance against the beveled face 48′ of the second stylet 16 to the advancement of the needle 112 through the human tissue 60 applies the bending force F_{B} against the beveled face 48′. The bending force F_{B} applied against the beveled face 48′ is thus exerted to the tip portion 128 of the needle body 120. Thus, as the stylet shank 40′ of the second stylet 16 is pliable, lateral deflection of the tip portion 128 of the needle body 120 will occur due to the bending of the flexible hinge portion 132 against the beveled face 48′ of the pliable stylet 16 as the needle assembly 10 advances through the tissue 60, as illustrated in FIG. 9B.

In operation, as the user steers the needle body 120 toward a target area 70 of the human tissue 60, over-shooting of the target area 70 may occur. For example, as shown in FIG. 9B, the tip portion 128 of the needle body 120 clearly misses the target area 70. In this case, a trajectory of the tip portion 128 of the needle body 120 needs to be re-adjusted. To correct the trajectory of the needle 112 in case of over shooting the target area 70, the soft stylet 16 can be just turned to an appropriate angle so as to change orientation of the beveled face 48′ thereof relative to the target area 70, as shown in FIG. 9C. Advancing the needle 112 further through the human tissue 60 would make the tip portion 128 of the needle body 120 move against the beveled face 48′ of the stylet 16, and therefore correcting the trajectory.

One of ordinary skill in the art would understand that the softer the stylet shank 40′ of the second stylet 16, the bigger the angle of deflection of the tip portion 128 relative the main portion 130 of the needle 112. Therefore the stylets 14 and 16 of the needle assembly 110 control an angle of inclination of the tip portion 128 of the needle body 120 relative to the main portion 130 thereof.

Preferably, the second (pliable) stylet 14 is inserted into the needle body 120 of the needle 112 so that the beveled face 48′ of the stylet shank 40′ completely extends outwardly from the distal face 134 of the needle body 120, as shown in FIG. 8. Alternatively, the beveled face 48′ of the second (pliable) stylet shank 40′ may only partially extend outwardly from the distal face 134 of the needle body 120, as shown in FIG. 10. It will be appreciated that the angle of inclination of the tip portion 128 of the needle body 120 relative to the main portion 130 thereof can be controlled by adjusting the degree of extension of the beveled face 48′ of the stylet 16 outwardly from the distal face 134 of the needle body 120. Specifically, the bigger portion of the beveled face 48′ of the stylet shank 40′ extending outwardly from the distal face 134 of the needle body 120, the bigger the angle of deflection of the tip portion 128 relative the main portion 130 of the needle 112. In other words, the needle assembly 10 shown in FIG. 8 provides bigger deflection of the tip portion 128 of the needle 112 than the needle assembly shown in FIG. 10.

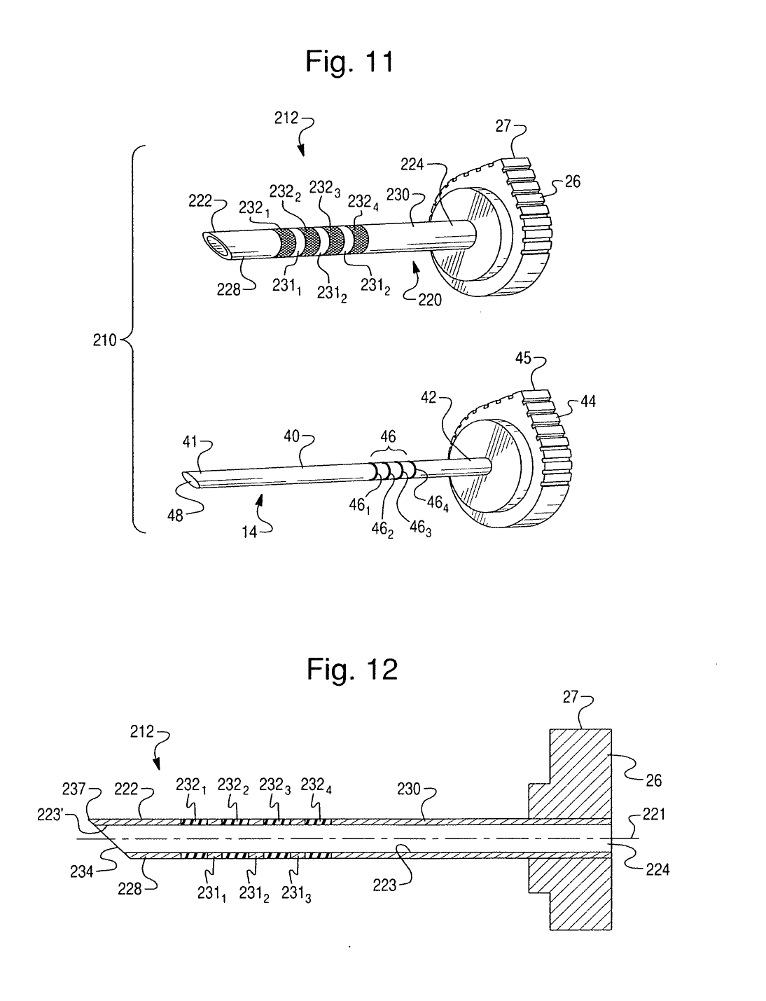

FIGS. 11, 12 and 13A-13C illustrate a needle assembly 210 according to a third exemplary embodiment of the present invention. Components, which are unchanged from the previous exemplary embodiments of the present invention are labeled with the same reference characters. Components, which function in the same way as in the first exemplary embodiment of the present invention depicted in FIGS. 1-6C are designated by the same reference numerals to which 200 has been added, sometimes without being described in detail since similarities between the corresponding parts in the two embodiments will be readily perceived by the reader.

The needle assembly 210 according to the third exemplary embodiment of the present invention comprises a needle 212, and a rigid elongated stylet 14. As illustrated in FIGS. 11 and 12, the needle 212 has an elongated tubular, preferably cylindrical, needle body 220 having open distal and proximal ends 222 and 224, respectively, and a needle hub (or needle handle) 26 provided at the proximal end 224. The needle body 220 has a central longitudinal axis 221 and includes a rigid tip portion 228 defining the distal end 222 thereof, a rigid main portion 230 defining, the proximal end 224, and a plurality of flexible hinge portions 232₁, 232₂, 232₃ and 232₄ connecting the tip portion 228 to the main portion 230 so as to enable the tip portion 128 to bend relative to the main portion 130. Rigid connecting tubes 231₁, 231₂ and 231₃ interconnect the flexible hinge portions 232₁, 232₂, 232₃ and 232₄. Preferably, the rigid tip portion 128, the rigid main portion 130 and the connecting tubes 231₁, 231₂ and 231₃ of the needle body 220 are made of a conventional steel material of medical needles.

Preferably, the flexible hinge portions 232₁, 232₂, 232₃ and 232₄, and the connecting tubes 231₁, 231₂ and 231₃ are substantially structurally identical in this embodiment. In view of these similarities and in the interest of simplicity, the following discussion will use a reference numeral in brackets without a letter to designate an entire group of substantially identical structures. For example, the reference numeral [232] will be used when generically referring to the flexible hinge portions 232₁, 232₂, 232₃ and 232₄ rather than reciting all four reference numerals. The flexible hinge portions [232] are made of any appropriate pliable material, such as pliable plastic material of conventional catheters. Distal ends of the hinge portions [232] are connected to corresponding ends of the tip portion 228, the connecting tubes [231] and the main portion 230 in any appropriate manner known in the art, such as by adhesive bonding. Each of the flexible hinge portions [232] is smaller than the hinge portion 32 in the first embodiment of the present invention.

Further according to the third exemplary embodiment of the present invention, the distal end 222 of the tip portion 228 of the needle body 220 is beveled at an acute angle to the longitudinal axis 221 of the needle body 220 to form a beveled (oblique) planar oval face 234 extending across a width of the needle body 220. The beveled face 234 extends from an end 236 along a length of the needle body 220 to a tip 237 of the tip portion 228 of needle body 220. The tip 237 is defined as an outermost point of the needle body 220. The acute angle between the beveled face 234 and the longitudinal axis 221 of the needle body 220 is preferably in the range of 30-60°.

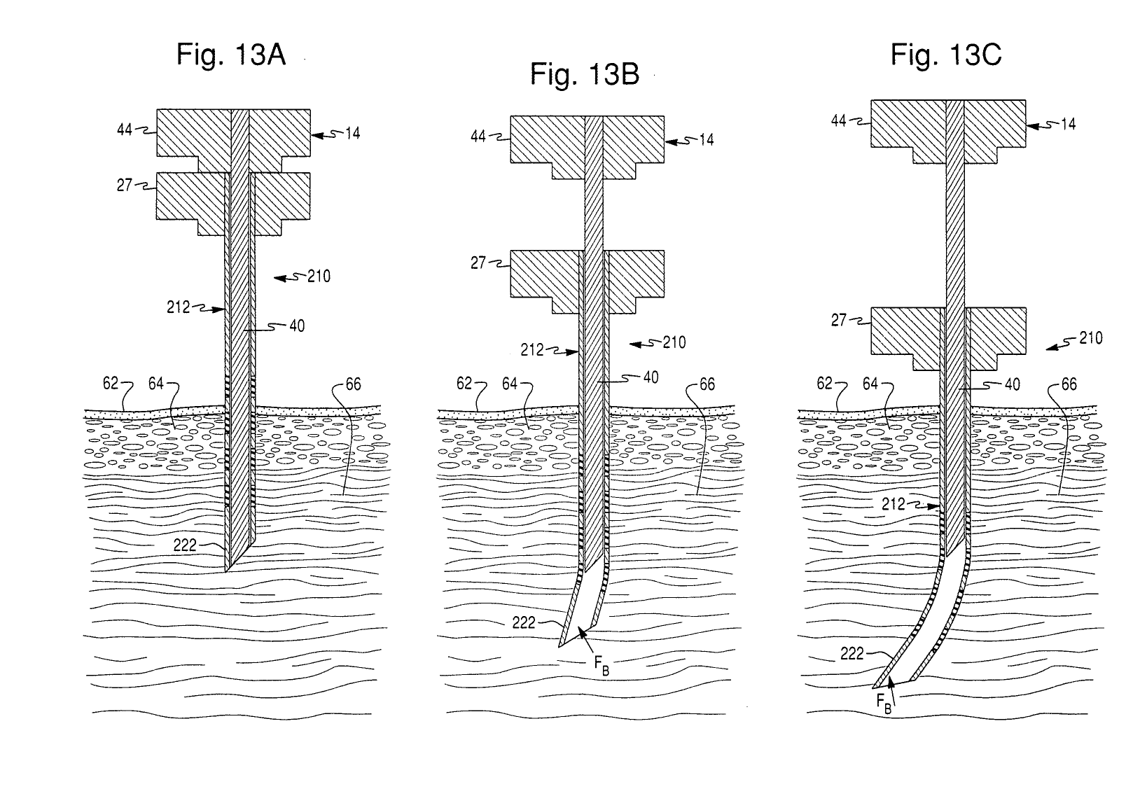

In operation, first, the rigid stylet 14 is inserted into the needle body 220 of the needle 212 so that the beveled face 48 of the stylet shank 40 is adjacent to the beveled face 234 of the needle body 220, shown in FIG. 13A. Next, the needle assembly 210 is inserted through the skin 62 and advanced to the appropriate depth within the human tissue 60. Then, if the need to change the direction of the tip portion 228 of the needle body 20 arises (in order to steer the needle 212 in a desired direction), the rigid stylet 14 is partially pulled back from the needle body 220 until the distal end 41, hence the tip 50, of the stylet shank 40 of the stylet 14 lies in one of the flexible hinge portions [232] of the needle body 220. It will be appreciated that further advancing of the needle 212 will cause the deflection (or bending) of the tip portion 228 of the needle body 220 against the beveled face 234 of the needle 212, as shown in FIGS. 13B and 13C. Specifically, as the needle assembly 210 advances through the human tissue 60, resistance to the advancement of the needle 212 through the human tissue 60 applies a bending force F_{B} against an internal surface 223 of the beveled distal end 222 of the needle body 20 adjacent to the tip 237 thereof. The stylet 14 is graded with depth markers/grading 46 on the proximal end 36 of the stylet shank 40 of the rigid stylet 14 (shown in FIG. 11) so that the user (e.g. injectionist) knows how many hinges he or she is activating. For instance, pulling the stylet 14 back to a first grading 46₁ would allow a small bend to occur. Pulling the stylet back to second, third and fourth gradings 46₂, 46₃ and 46₄ will allow a progressively larger bending to occur. Therefore, the stylet 14 of the needle assembly 210 allows to variably control the angle of inclination of the tip portion 228 of the needle body 220 relative to the main portion 230 thereof.

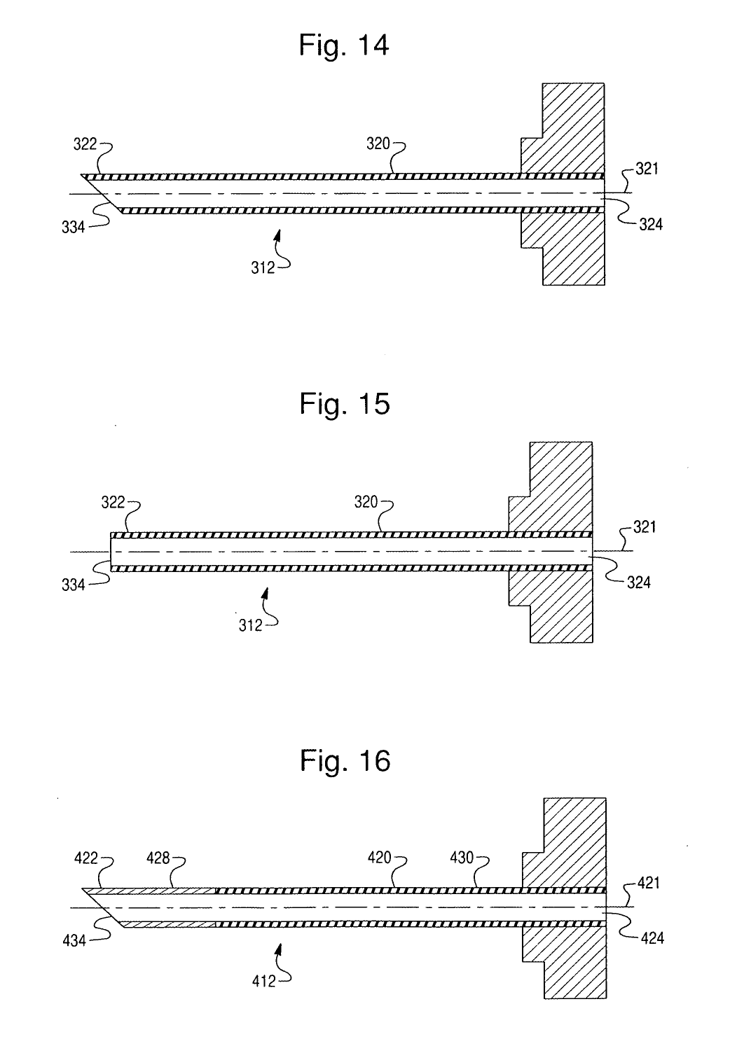

FIGS. 14 and 15 illustrate a needle assembly 3 10 according to a fourth exemplary embodiment of the present invention. Components, which are unchanged from the previous exemplary embodiments of the present invention are labeled with the same reference characters. Components, which function in the same way as in the first to third exemplary embodiments of the present invention depicted in FIGS. 1-13C are designated by the same reference numerals, sometimes without being described in detail since similarities between the corresponding parts in the two embodiments will be readily perceived by the reader.

The needle assembly 310 according to the third exemplary embodiment of the present invention comprises a needle 312, shown in FIG. 14, and a rigid elongated stylet 14. Alternatively, the needle assembly 310 may further include a soft elongated stylet 16. As illustrated in FIG. 14, the needle 312 has an elongated tubular, preferably cylindrical, bendable needle body 320 having open distal and proximal ends 322 and 324, respectively, and a needle hub (or needle handle) 26 provided at the proximal end 324. The needle body 320 has a central longitudinal axis 321 and is made of any appropriate pliable material, such as pliable plastic material of conventional catheters.

Further according to the fourth exemplary embodiment of the present invention the distal end 322 of the needle body 320 is beveled at an acute angle to the longitudinal axis 321 of the needle body 320 to form a beveled (oblique) planar oval face 334 extending across a width of the needle body 320. Alternatively, as shown in FIG. 15, the distal end 322 of the needle body 320 is symmetrical and without a bevel. In other words, the distal face 334 of the needle body 320 is substantially orthogonal to the longitudinal axis 321 thereof the needle body 320.

FIG. 16 illustrates a needle assembly 410 according to a fifth exemplary embodiment of the present invention. Components, which are unchanged from the previous exemplary embodiments of the present invention, are labeled with the same reference characters. Components, which function in the same way as in the first to fourth exemplary embodiments of the present invention depicted in FIGS. 1-15 are designated by the same reference numerals, sometimes without being described in detail since similarities between the corresponding parts in the two embodiments will be readily perceived by the reader.

The needle assembly 410 according to the fifth exemplary embodiment of the present invention comprises a needle 412, a rigid elongated stylet 14, and a soft elongated stylet 16. As illustrated in FIG. 16, the needle 412 has an elongated tubular, preferably cylindrical, bendable needle body 420 having open distal and proximal ends 422 and 424, respectively, and a needle hub (or needle handle) 26 provided at the proximal end 424. The needle body 420 has a central longitudinal axis 321 and is made of any appropriate pliable material, such as pliable plastic material of conventional catheters.

The needle body 420 has a central longitudinal axis 421 and includes a rigid, penetrating tip portion 428 defining the distal end 422 thereof, and a bendable (pliable) main portion 430 defining the proximal end 424. The tip portion 428 is connected to the main portion 430 in any appropriate manner known in the art, such as by adhesive bonding, so as to enable the tip portion 428 to bend relative to the main portion 430. Preferably, the rigid tip portion 428 is made of a conventional steel material of medical needles, while the bendable main portion 430 is made of any appropriate pliable material, such as pliable plastic material of conventional catheters:

Further according to the fifth exemplary embodiment of the present invention, the distal end 422 of the needle body 420 is beveled at an acute angle to the longitudinal axis 421 of the needle body 320 to form a beveled (oblique) planar oval face 434 extending across a width of the needle body 420. Alternatively, the distal end 422 of the needle body 420 may be symmetrical and without a bevel. In other words, the distal face 434 of the needle body 420 may be substantially orthogonal to the longitudinal axis, 421 thereof the needle body 420.

Therefore, the present invention provides a novel needle assembly allowing for controlled steering of the needle assembly as it advances through the biological tissue in order to position a distal end of a needle exactly where needed as opposed to where is accessible with rigid straight or curved needles.

The foregoing description of the preferred embodiments of the present invention has been presented for the purpose of illustration in accordance with the provisions of the Patent Statutes. It is not intended to be exhaustive or to limit the invention to the precise forms disclosed. The embodiments disclosed hereinabove were chosen in order to best illustrate the principles of the present invention and its practical application to thereby enable those of ordinary skill in the art to best utilize the invention in various embodiments and with various modifications as suited to the particular use contemplated, as long as the principles described herein are followed. This application is therefore intended to cover any variations, uses, or adaptations of the invention using its general principles. Further, this application is intended to cover such departures from the present disclosure as come within known or customary practice in the art to which this invention pertains. Thus, changes can be made in the above-described invention without departing from the intent and scope thereof. It is also intended that the scope of the present invention be defined by the claims appended thereto.

What is claimed is:

1. A needle assembly comprising:
a needle including an elongated tubular needle body having open distal and proximal ends, said needle body including a tip portion defining said distal end and a main portion defining said proximal end, said tip portion bendable relative to said main portion; and
an elongated stylet having a solid shank complementary to said tubular needle body and slidably movable therewithin substantially coaxially thereto in the direction between said proximal and distal ends of said needle body, said stylet shank having solid distal and proximal ends;
at least one of said distal end of said needle body and said distal end of said stylet shank being beveled at an angle to a longitudinal axis of corresponding one of said needle body and said stylet shank;
said at least one beveled distal end of one of said needle body and said stylet shank exerting a bending force to said tip portion of said needle body against said beveled distal end during advancement of said needle body through a biological tissue;
said stylet controlling an angle of inclination of said tip portion of said needle body relative to said main portion thereof in order to steer said needle during advancement thereof through said biological tissue.

2. The needle assembly as defined in claim 1, wherein said tip and main portions of said needle body are made of a rigid material, and wherein said needle body further includes at least one bendable hinge portion connecting said tip portion to said main portion so as to enable said tip portion to bend relative to said main portion.

3. The needle assembly as defined in claim 2, wherein said at least one bendable hinge portion of said needle body is made of a pliable material.

4. The needle assembly as defined in claim 2, wherein said tip and main portions of said needle body are made of a non-pliable material.

5. The needle assembly as defined in claim 1, wherein said at least one bendable hinge portion includes a plurality of bendable hinge portion connected to each other through rigid connecting tubes.

6. The needle assembly as defined in claim 1, wherein said stylet is made of a bendable material.

7. The needle assembly as defined in claim 1, wherein said stylet is made of a rigid, non-pliable material.

8. The needle assembly as defined in claim 1, wherein said solid shank of said stylet is provided with at least one depth marker adjacent to a proximal end of said shank.

9. The needle assembly as defined in claim 1, wherein said solid distal end of said stylet shank is beveled at an angle to a longitudinal axis of said stylet shank to form an oblique face extending across a width of said stylet shank; said beveled face of said shank exerts said bending force to said tip portion of said needle body applied against said beveled face when said needle assembly advances through said biological tissue.

10. The needle assembly as defined in claim 9, wherein said stylet further includes a stylet hub provided at said proximal end of said stylet shank.

11. The needle assembly as defined in claim 10, wherein said stylet hub includes a pointed ridge oriented in alignment with a tip of said oblique face of said distal end of said stylet shank for allowing a tactile confirmation of a bevel direction of said distal end of said stylet shank.

12. The needle assembly as defined in claim 1, wherein said distal end of said needle body is beveled at an angle to a longitudinal axis of said needle body to form an oblique face extending across a width of said needle body;
said beveled distal end of said needle body exerts said bending force to said tip portion of said needle body against said beveled face during advancement of said needle body through said biological tissue.

13. The needle assembly as defined in claim 12, wherein said needle further includes a needle hub provided at said proximal end of said needle body.

14. The needle assembly as defined in claim 13, wherein said needle hub includes a pointed ridge oriented in alignment with a tip of said oblique face of said distal end of said needle body for allowing a tactile confirmation of a bevel direction of said distal end of said needle body.

15. The needle assembly as defined in claim 1, wherein said needle body is made of a pliable material.

16. The needle assembly as defined in claim 1, wherein said tip portion of said needle body is made of a rigid material, and wherein said main portion is made of a pliable material so as to enable said tip portion to bend relative to said main portion.

17. The needle assembly as defined in claim 1, wherein said needle further includes a needle hub provided at said proximal end of said needle body.

18. The needle assembly as defined in claim 17, wherein said needle hub is made of a transparent material.
